(19) United States
(12) Patent Application Publication
(10) Pub. No.: US 2014/0228634 A1
(43) Pub. Date: Aug. 14, 2014

(54) AUTOMATED ACTUATOR FOR SPRING BASED MULTIPLE PURPOSE MEDICAL INSTRUMENTS

(71) Applicant: David S. ZIMMON, Port Washington, NY (US)

(72) Inventor: David S. ZIMMON, Port Washington, NY (US)

(21) Appl. No.: 14/258,117

(22) Filed: Apr. 22, 2014

Related U.S. Application Data
(62) Division of application No. 12/928,527, filed on Dec. 14, 2010.
Application No. 12/928,527 is a continuation-in-part of application No. 11/435,058, filed on May 16, 2006, now abandoned.
(60) Provisional application No. 61/284,230, filed on Dec. 14, 2009.

(51) Int. Cl. A61B 1/00; A61B 17/3205; A61B 17/29; A61B 1/018; A61B 10/04

(52) U.S. Cl. CPC A61B 1/00133; A61B 1/00105; A61B 1/018; A61B 10/04; A61B 17/29; A61B 17/32056; A61B 2010/0208
USPC 600/106

(57) ABSTRACT

An automated switch and driver for an open biased spring-based multiple purpose end effector of a medical instrument to be used by a single operator without assistance for biopsy, clipping, clamping, grasping, snaring, cutting, dissecting or other operative functions with electrical connections for cautery or hot biopsy used independently or combined with a rigid or flexible endoscope of any size. The driver mechanism may be a spring(s), gear(s), electrical solenoid or motor, air or hydraulic pressure activated piston or a combination thereof. Automated action may be initiated by voice, hand or foot switch. The switch and driver mechanism may be disposable, attached to or separable from the operative instrument or permanently attached to be reusable. The automated end effector instrument may be combined with or incorporated into an endoscope.

### CROSS-REFERENCE TO RELATED APPLICATIONS

This application is a divisional of U.S. patent application Ser. No. 12/928,527, which claims priority under 35 U.S.C. §119(e) of U.S. Provisional Application Ser. No. 61/284,230, filed on Dec. 14, 2009. U.S. patent application Ser. No. 12/928,527 is also a continuation-in-part under 35 U.S.C. §120 of U.S. patent application Ser. No. 11/435,058, filed on May 16, 2006.

1. Field of the Invention

The present invention relates to an automated switch and driver for use with an open biased spring-based end effector operative instrument and endoscope for medical procedures including biopsy, clipping, clamping, grasping, snaring, cutting, dissecting or other actions.

2. The Prior Art

It is often necessary to perform operative procedures through small access sites deep into a lumen or a cavity. These operations are performed by insertion of flexible or rigid instruments using palpation, visual, endoscopic or radiologic imaging control including fluoroscopy, magnetic resonance or ultrasound. The shaft or handle is used to advance or withdraw the instrument by hand. The handle pulls a long wire that pivots a jaw-fulcrum operated end effector. Simultaneous manipulation of endoscope, instrument position and end effector handle mechanism requires the coordinated action of two or more trained operators.

For example, during endoscopic biopsy, the insertion and forward or back movement of the endoscope is performed with the endoscopists right hand, while the left-right and up-down are operated with the left. Additionally there are air inflation and suction controls and an instrument elevator also operated by the left hand. Operating the end effector requires coordination with a skilled assistant. The assisting staff is exposed to infectious material and radiation requiring specialized protective clothing when fluoroscopy is used. Sterile clothing is required for operative field sterility. Disposable clothing is required to limit contamination in colonoscopy. Additionally there is the cost of the assistant and the additional operating room space required for assisting staff. These requirements complicate the operative procedure, add cost and risk if any of the protective measures fail. For the patient, this complexity prolongs the procedure, increases the anesthesia duration, cost and risk of operative error.

U.S. Pat. No. 5,431,645 to Smith et al. describes a remotely activated endoscopic tool such as a biopsy forceps that comprises a driver, coupler, power source, interface and a sensing/feedback interface to monitor actuator tension with multiple sources of motive force and controllers. U.S. Pat. No. 6,817,973 B2 to Merril at al. describes an apparatus for controlling force for manipulating medical instrument for the passage and withdrawal of an instrument through the channel of an endoscope. After positioning the instrument, the operator takes control for the necessary fine operative movements. U.S. Patent Application Publication No. 2007/0185377 to Murakami at al. describes an endoscopic System for instrument introduction through the endoscope channel and end effector operation with sensors for both introduction and end effector operation that feed back to controllers, displays and recording memory modules. Nevertheless, the operator must manipulate a remotely activated instrument while visually monitoring the end effector and operating the endoscope with its multiple tasks.

Jaw fulcrum end effectors need complex operational systems because of the frictional resistance raised to the fourth power of each curve traversed by the long wire(s). Wire tension to push open or pull closed the end effector straightens the wire, instrument shaft and flexible endoscope. Wire binding at curves with shaft collapse is prevented by stiffening the instrument sheath. Frictional resistance requires complex sensors and feedback controls to imitate the variable force of an operator's eye and hand. Complex multiple component systems are difficult to incorporate into slender long endoscopic instruments or endoscopes. U.S. Pat. No. 5,782,747 to Zimmon, which is herein incorporated by reference, describes a spring based multiple purpose medical instrument for biopsy, clamping, cutting, grasping, dissecting and other end effector actions. The device is opened by either retracting the housing or by extending the open biased spring arms beyond the housing. For closure, the housing is extended or spring arms pulled to slide into the housing. Driver wire And housing stops limit spring arm extension and retraction.

Spring based end effector movement resistance is reduced by a housing cam guide ramp that stabilizes flat spring arms as they slide within the housing. The distance between the spring based end effector arms is set by the housing guide cam and is proportional to the extension of the spring from the housing and opening angle of the arms. Short arm movement within the housing minimizes the force needed to open or close the end effector and backlash. This design minimizes wire friction and shaft straightening when the instrument is positioned through the endoscope for operation or used independently.

Consequently, a brief driver force opens or closes a 230 cm spring-based end effector when passed through an endoscope that traverses multiple curves.

This design allows precise mechanical driver control of the end effector in intermediate positions between open and closed. When the spring-based instrument is partly or completely opened or closed, the distance between the arms is maintained by housing guide pressure on the arms. A ratchet created between the spring arms and housing guide cam increases position stability when necessary. The force applied to an object gripped between the arms is the tension of the spring arms. Closing force is similarly limited by the arm's spring tension until the arms completely close on entering the housing. Absent driver movement, the open biased spring-based end effector is mechanically fail-safe from opening or closing without operator attention or muscular coordination. A spring-based end effector is precisely controlled by driver movement yet still mechanically stable when movement is absent. Position sensors, feedback controls or monitors are unnecessary. The actuating mechanism is a switch operating the automated driver device to push or pull the end effector with a long or short wire at the speed and short linear distance required by the specific end effector. This operational simplicity facilitates automation of an open biased spring based end effector and combining or incorporating into an endoscope.

Control of opening, intermediate positions and closing of a spring based end effector is important during grasping as for tissue retraction where end effector closing pressure must be maintained for prolonged periods. When performed by hand this is tedious and may subject the tissue to excessive compression resulting in tissue damage or grasp failure with loss of the operative position. Graded closure is needed for cutting and clamping, polyp removal with a snare using cautery or dissection.

Dissection is performed by advancing the housing followed by opening the spring arms. The lateral dissection distance is distance between the spring arms. The dissecting force is the opening force of the spring action applied by extending the spring arms or withdrawing the housing. The lateral distance dissected is the extension of the spring arms from the housing constraint. Automation of other end effector assemblies is similarly feasible with consideration given to the specific mechanical properties of the end effector.

In U.S. Pat. Nos. 5,980,468 and 6,071,248, both to Zimmon, which are herein incorporated by reference, utilize the lateral biopsy device disclosed in U.S. Pat. No. 5,685,320 and spring based multi-purpose medical instrument in U.S. Pat. No. 5,980,468, both to Zimmon which are also herein incorporated by reference, to collect and process multiple biopsy specimens in a single pass to the biopsy site with or without an endoscope. These methods overcome the prior necessity of removing the biopsy instrument to collect and segregate each specimen for site identification after each biopsy. This necessity minimized the usefulness of prior inventions (Smith et al. U.S. Pat. No. 5,431,645; Merril et al. U.S. Pat. No. 6,817,973 B2) for remotely activating biopsy devices since the entire apparatus must be removed and replaced to collect and identify each or only a few specimens.

In U.S. patent application Ser. No. 11/435,058 to Zimmon, published as US 2007-0270894 A1, which is herein incorporated by reference, a hand operated spring based biopsy instrument is combined with the endoscope to facilitate the use and reduce the cost of both. Automating the performance of biopsy, clip, clamp, snare, cutting, dissecting and other functions further increases the efficiency and reduces the cost of these devices used in combination with an endoscope.

Although the prior art has made safe efficient operations deep within the patient possible, the need for additional improvements remains.

### SUMMARY OF THE INVENTION

It is therefore an object of the present invention to provide an automated switch and driver for an open biased spring based multiple purpose medical operative instrument actions of biopsy, clipping, clamping, grasping, snaring, cutting, and dissecting or other functions. A further object of the present invention is to incorporate or combine the automated spring based multiple purpose instruments into endoscopes fully or in part. The automated switch, driver, connecting assembly and end effector may be fully or in part within or outside the endoscope as permanent or removable and replaceable.

The present invention therefore comprises an operative device having a handle and a rigid or flexible shaft connected to the handle, a switch, driver and a connected open biased spring-based end effector for biopsy, clipping, clamping, grasping, snaring, dissecting or cutting. The spring based end effector is disposed inside the operative device housing and is movable by the driver into and out of the operative device housing. There is an automated switch operating the driver, which is adapted to move the spring based end effector into and out of the housing. The switch mechanism connected to the automated driver device operates the automated spring based operative device without or with the use of the operator's hands. This switch mechanism can be a foot switch, a switch on the shaft or handle, a voice activated switch or any other suitable mechanism. The automated operative device may combined with or incorporated into an endoscope in a variety of configurations.

The automated driver device can be a spring, electrical solenoid, electric motor, air or hydraulic pressure activated piston, gear, or any combination thereof.

In a preferred embodiment, the end effector is any open biased spring based multiple purpose device having spring arms, a housing guide ramp, and a ratchet between the spring arms and housing guide ramp, for biopsy, clipping, clamping, grasping, snaring, cutting, dissecting or other functions. Moving the end effector out of the housing opens the spring arms, moving the end effector into the housing pushes the spring arms closed by interior walls of the housing. There can be a removable and replaceable biopsy storage cassette disposed within the spring arms, so that multiple biopsies collected by the cutting jaws are collected in the storage cassette in the order of acquisition.

In another embodiment of the invention, the device comprises an endoscope having a shaft with an instrument channel, a handle on one end of the endoscope and a spring-based end effector for biopsy, clipping, clamping, grasping, snaring, dissecting or cutting, connected to the endoscope. There is an automated driver device connected to the end effector and adapted to operate the end effector; an automated driver mechanism connected to the automated driver device for operating the automated end effector without the use of the operator's hands.

In one embodiment, the end effector is disposed within a distal end of the endoscope in the instrument channel, and the automated driver device is connected to the end effector by a narrow shaft introduced and removed through the instrument channel within the endoscope or attached to the outside of the endoscope.

In another embodiment, the automated switch device is disposed in the endoscope handle and there is a removable and replaceable shaft connected outside the endoscope to a distal endoscope section. The end effector switch passes through this removable and replaceable shaft and is connected to the automated driver device via a wire running through the shaft. Preferably, the handle and the automated switch device are separable from the shaft and the end effector, and are reusable. The end effector could be separable from the shaft and be replaceable as well.

In another embodiment, the automated switch and driver devices are disposed within the endoscope handle and the end effector is disposed within a distal endoscope section and is removable and replaceable. The driver device is connected to the end effector via a wire passing down the endoscope shaft to the end effector.

In yet another embodiment, the automated switch and driver devices are disposed within the handle and the end effector is attached to a cover of the endoscope and is removable and replaceable. The automated driver device is connected to the end effector by a wire that passes inside the endoscope shaft to the end effector.

In a further embodiment, the automated driver device is incorporated into a distal section of the endoscope and the end effector is attached outside of the endoscope and is removable and replaceable.

In a further embodiment, the automated driver device is attached to an outside of the endoscope.

The actuating mechanism can be a foot switch, a control on the shaft or handle, a voice activated switch or any other suitable mechanism.

There can be a removable and replaceable biopsy cassette mounted within the end effector, such that biopsies collected by the end effector are automatically fed into the removable replaceable biopsy cassette in the order of collection.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

FIG. 1 shows a side view of one embodiment of an end effector assembly according to the invention;

FIG. 2 shows an exploded view of the end effector assembly shown in FIG. 1;

FIG. 3 shows a longitudinal view of a spring-based multiple biopsy end effector assembly with the jaws fully extended to the driver stop for biopsy;

FIG. 3a shows the end effector assembly of FIG. 3 capturing multiple biopsies and placing them in a removable biopsy cassette;

FIG. 4 shows a longitudinal view of a spring-based multiple biopsy end effector assembly fully retracted to the housing stop to move the specimen(s) into the storage housing;

FIG. 5 shows a longitudinal view of an endoscope with an automated switch and driver device connected outside the endoscope handle and of a disposable replaceable multiple biopsy end effector passed through an endoscopic operative device instrument channel;

FIG. 6 shows a longitudinal view of an automated switch and driver mechanism connected to the distal segment of an endoscope via a separate shaft to operate a removable replaceable multiple biopsy end effector that is disposed outside the endoscope;

FIG. 7 shows a longitudinal view of the endoscope having an automated switch and driver mechanism disposed within the handle with a separable shaft and a removable replaceable spring-based end effector;

FIG. 8 shows a longitudinal view of an endoscope with an switch and electrical connection in the endoscope handle passing within the endoscope shaft to operate an automated driver device and end effector in the distal segment of an endoscope;

FIG. 9 shows the distal end of an endoscope shaft with the end effector assembly disposed outside the endoscope shaft;

FIG. 10 shows the distal end of an endoscope shaft with the end effector assembly disposed on the cover of the shaft and connected via a separate external actuating shaft;

FIG. 11 shows an endoscope with an automated driver mechanism in the distal segment of an endoscope and an external switch and removable replaceable spring-based end effector attached to the outside of the instrument end;

FIG. 12a shows a longitudinal view of a blade anvil open biased spring based cutting tool extended from the shaft housing;

FIG. 12b shows a longitudinal view of an open biased spring based detachable clip extended from the shaft housing;

FIG. 12c shows a longitudinal view of an open biased spring based clamp with cautery extended from the shaft housing;

FIG. 12d shows a longitudinal view of an open biased spring based dissecting tool with cautery extended from the shaft housing;

FIG. 12e shows a longitudinal view of an open biased spring based snare extended from the housing to capture a polyp;

FIG. 12f shows a longitudinal view of a snare partially closed after capturing a polyp; and

FIG. 12g shows a longitudinal view of an open biased spring based grasper with ratchet extending from the shaft housing.

### DETAILED DESCRIPTION OF THE EMBODIMENTS

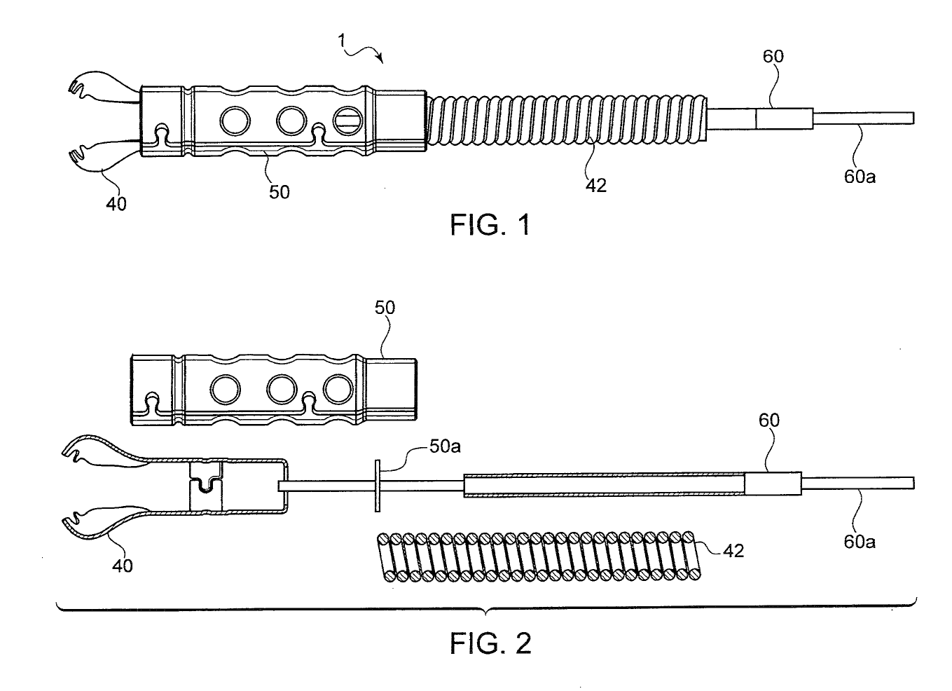

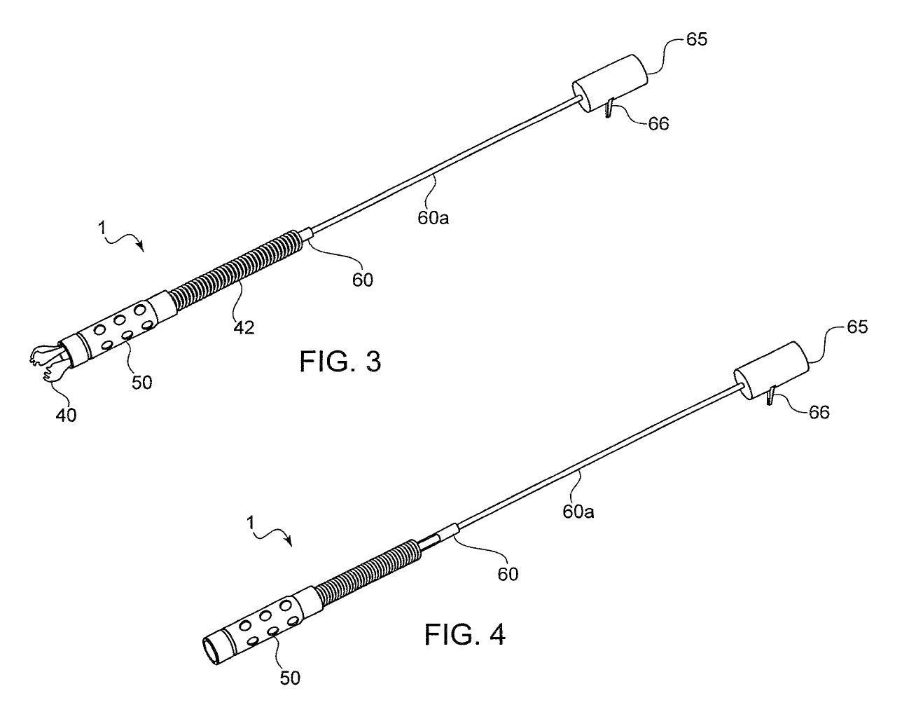

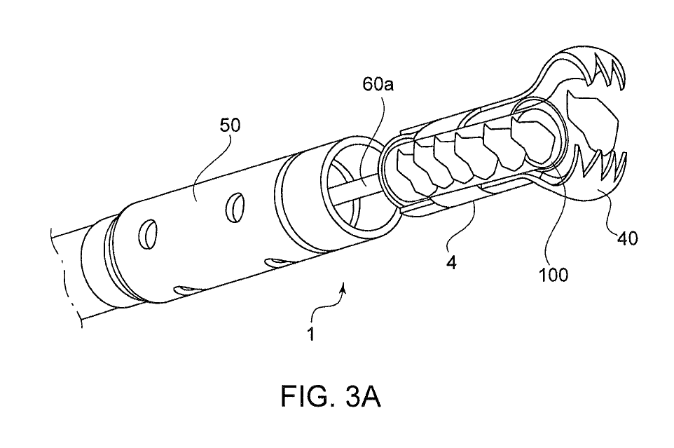

The device shown in FIGS. 1-4 comprises a spring-based biopsy end effector generally designated as 1 with a driver wire 60a connected to spring arms 40 in a housing 50. A cable or other spacer 42 connected to the housing 50 keeps spring arms 40 in the correct position relative to the housing 50 as shown in FIGS. 3 and 3a until driver wire 60a is pulled to retract arms 40 within housing 50, to capture a biopsy 100, as shown in FIGS. 3a and 4. As shown in the exploded view in FIG. 2, there is an extension stop 60 and spacer that prevents overextension of spring arms 40, and a housing stop 50a which prevents over-retraction by the driver wire 60a.

According to the present invention, the pulling and releasing of wire 60a takes place via an automated driver mechanism 65 that is actuated by an automated switch device 66, shown in FIGS. 3 and 4. Driver mechanism 65 can comprise any suitable automatic force generator that can pull and push the driver wire. Suitable but not limiting examples are spring(s), an electric solenoid, an electric motor such as a stepper motor, an air or hydraulic pressure activated piston, gear(s), or a combination thereof. Connected to driver mechanism 65 is a switch device 66 for turning the driver mechanism on and off to pull and push driver wire 60a. Switch device 66 can be a mechanical switch, a foot pedal, or a voice-activated switch connected to driver mechanism 65. In this embodiment, switch device 66 is shown as a mechanical switch. Any other suitable type of switching device could also be used.

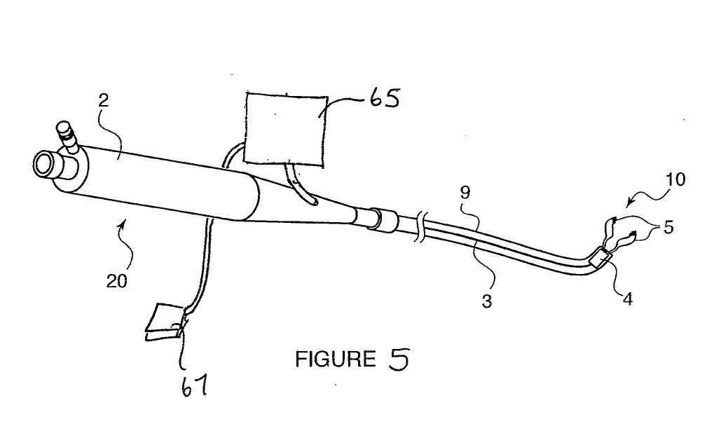

FIG. 5 shows a longitudinal view of another embodiment of the invention, in which the biopsy device is incorporated into an operative instrument such as an endoscope 20. Endoscope 20 has a handle 2 and an automated proximal driver device 65 through which actuating wire 3 passes via shaft 9 to spring arms 5 of multiple biopsy end effector 10. A removable replaceable biopsy cassette 4 is disposed at a distal end of shaft 9 for receiving biopsies captured by spring arms 5 in the order of collection. Automated driver mechanism 65 is connected to wire 3 via an external tube 7. Automate driver mechanism 65 generates a force for pushing the wire 6 to open spring arms 5 and pulling wire 3 to cause spring arms 5 to capture a biopsy and move it in to cassette 4. Automated driver mechanism 65 is connected to a switching device 67, which in this embodiment is a foot pedal, but could also be a hand or voice activated switch mechanism for turning automated driver mechanism on and off.

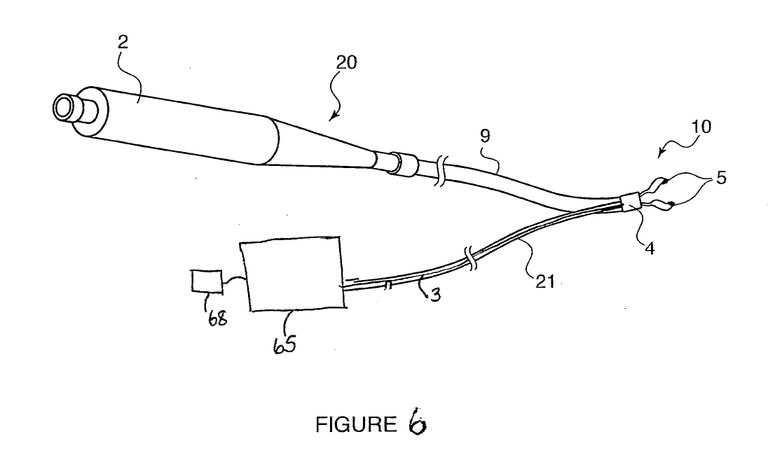

FIG. 6 shows a longitudinal view of endoscope 20 with automated external proximal driver mechanism 65 connected to spring arms 5 via a wire 3 through a separate actuating shaft 21. Automated driver mechanism 65 is connected to a voice activated switch 68 in this embodiment, but foot pedals or mechanical hand-activated switches could also be used. As discussed above, automated driver mechanism 65 can be any suitable mechanism for generating a motive force, such as a stepper motor, solenoid, hydraulic or pneumatic piston, etc. Speaking the required command in the vicinity of switch 68 causes driver mechanism 65 to pull the wire 3, causing spring arms 5 to retract into shaft 9 and deposit biopsies into cassette 4. As shown in FIG. 6, shaft 21 is detachable from endoscope 20 so that the driver mechanism 65 can be used on other endoscopes as well.

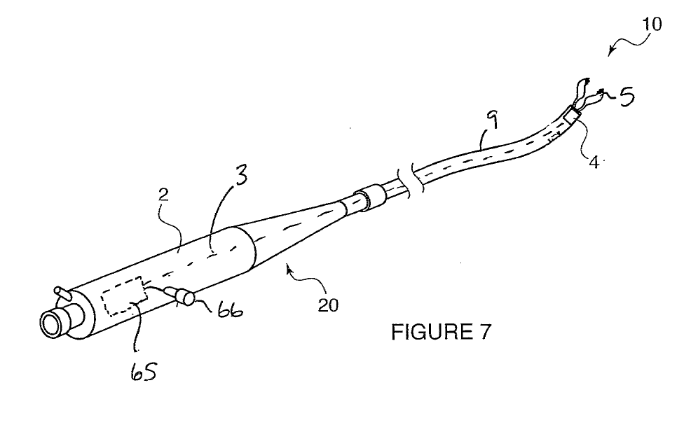

FIG. 7 shows a longitudinal view of endoscope 20 in which automated driver mechanism 65 is incorporated into handle 2 of endoscope 20. The switching device 66 is disposed on the exterior of handle 2 for actuation by the user. Switching device 66 could also be a voice activated switch incorporated in the handle, or a foot pedal connected to driver mechanism 65. Automated driver mechanism 65 is connected via wire 3 to spring arms 5 of the device. Shaft 9 and the entire end effector mechanism 10 are removable and replaceable.

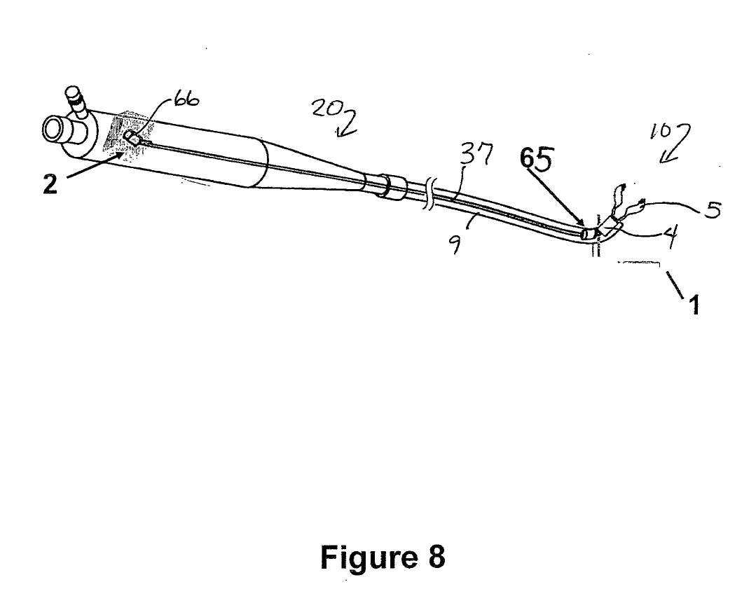

FIG. 8 shows a longitudinal view of endoscope 20 with switching device 66 incorporated in handle 2, and automated driver mechanism 65 disposed in a distal end of the endoscope shaft 9, adjacent the end effector assembly 10 with spring arms 5. Electrical connection 37 connects driver mechanism 65 with switching device 66 through the endoscope shaft 9. Shaft 9 and end effector assembly 10 are removable and replaceable.

FIG. 9 shows the distal end of endoscope shaft 9 having end effector assembly 10 disposed outside the endoscope shaft 9 and connected to the proximal automated driver mechanism via wire 3. Automated driver mechanism 65 can be incorporated into the endoscope handle as shown in FIG. S or can be disposed outside of the endoscope 20 as shown in FIGS. 5 and 6.

FIG. 10 shows the distal end of endoscope shaft 9 in which end effector assembly 10 is mounted to the exterior or cover of shaft 9, and has an exterior wire extending through shaft 21 for connection to the automated driver mechanism. Automated driver mechanism 65 can be incorporated into the endoscope handle as shown in FIG. 8 or can be disposed outside of the endoscope 20 as shown in FIGS. 5 and 6.

FIG. 11 shows the endoscope 20 having an automated driver mechanism 65 attached to the distal segment of the endoscope 20 and to an external removable replaceable spring based end effector assembly 10 having spring arms 5 attached to the outside of the endoscope end. Actuating shaft 21 connects automated driver mechanism 65 to switching device 66 with an electrical connection.

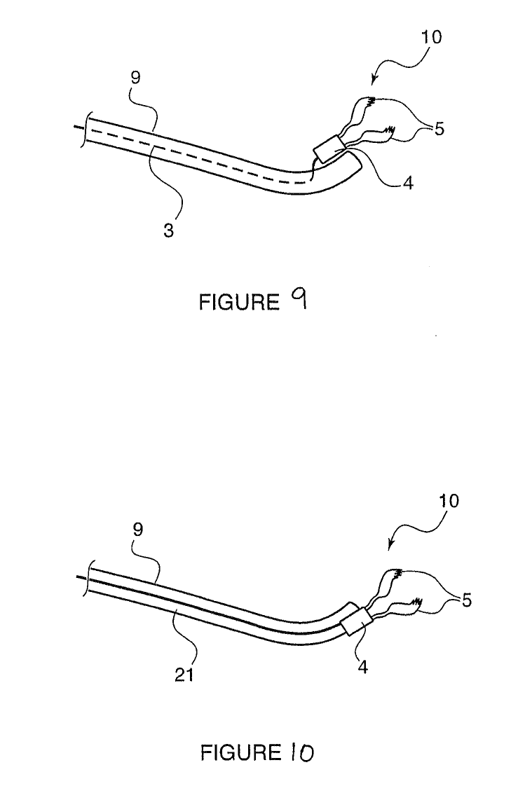

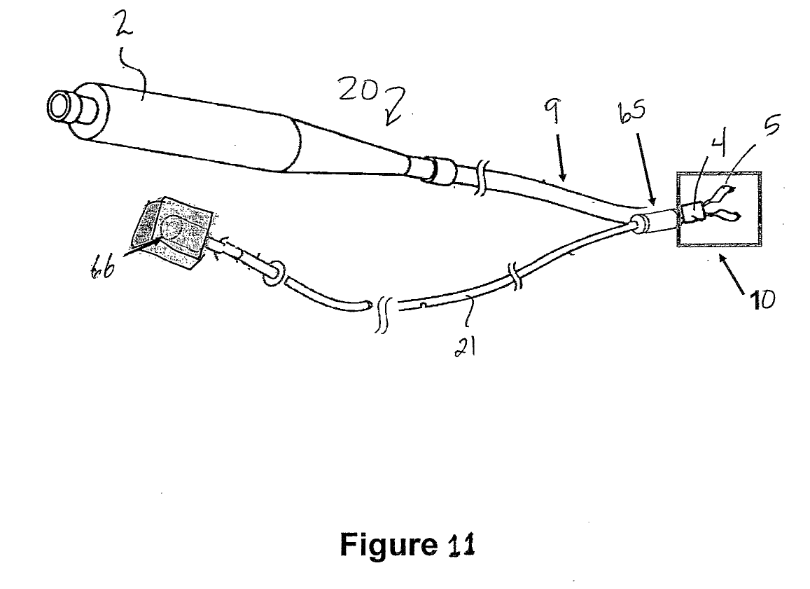

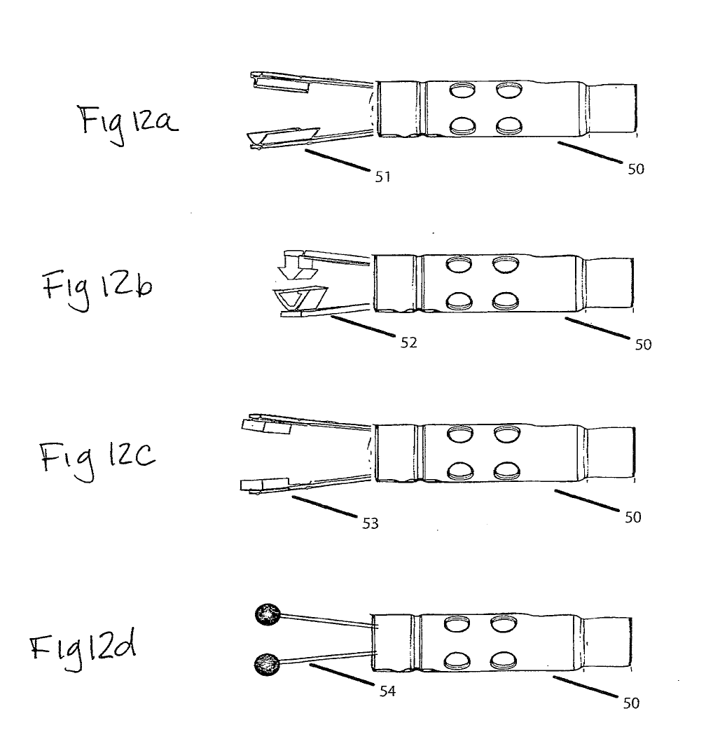

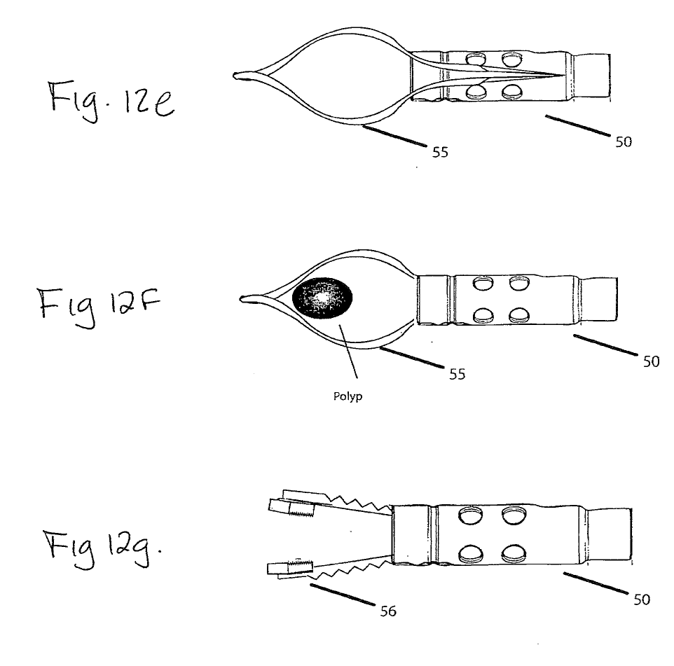

The embodiments shown in FIGS. 12a-12g show different variations of end effector assembly 10. Other variations could also be used. FIG. 12a shows a longitudinal view of a blade anvil spring based cutting tool 51 extended from housing 50.

FIG. 12b shows a longitudinal view of a detachable clip 52 extended from housing 50.

FIG. 12c shows a longitudinal view of a clamp 53 with cautery extended from housing 50.

FIG. 12d shows a longitudinal view of a dissecting tool 54 with cautery extended from housing 50.

FIG. 12e shows a longitudinal view of a snare 55 extended from housing 50 to capture a polyp, and FIG. 12f shows a longitudinal view of a snare 55 partially closed after capturing a polyp.

FIG. 12g shows a longitudinal view of a grasper 56 with a ratchet extending from housing.

The present invention provides a novel way for an operator to cut and collect biopsies without requiring an assistant, even while using an endoscope.

Accordingly, while only a few embodiments of the present invention have been shown and described, it is obvious that many changes and modifications may be made thereunto without departing from the spirit and scope of the invention.

1. An apparatus for performing a medical procedure, comprising:
a operative device having a handle and a rigid or flexible shaft connected to the handle;
a spring-based end effector for biopsy, clipping, clamping, grasping, snaring, dissecting or cutting, said spring based end effector being disposed inside the a housing of the operative device and being movable into and out of the housing, the end effector being a spring based multiple purpose device having spring arms connected to a driver wire, a housing for receiving the spring arms, a stop for maintaining the spring arms in an extended position;
an automated driver device connected to the end effector and being adapted to move the end effector into and out a housing of the operative device; and
a switch mechanism connected to the automated driver device for operating the automated driver device without the user of the operator's hands or an assistant, the switch mechanism being selected from the group consisting of a mechanical switch, a foot pedal, and a voice activated switch;
wherein actuating the switch mechanism causes the automated driver device to push the spring arms out of the housing and open the spring arms, or to pull the spring arms into the housing or push the housing over the spring arms, which causes the spring arms to be pushed closed by interior walls of the housing.

2. The apparatus of claim 1, wherein the automated driver device is selected from the group consisting of a spring, electrical solenoid, electric motor, air or hydraulic pressure activated piston, gear(s), and a combination thereof.

3. An automated operative instrument, comprising:
an endoscope having a shaft with an instrument channel;
a handle on one end of the endoscope;
a spring-based end effector for biopsy, clipping, clamping, grasping, snaring, dissecting or cutting, said end effector being connected to the endoscope;
an automated driver device connected to the end effector and being adapted to operate the end effector; and
a switch mechanism connected to the automated driver device for operating the automated driver device without an assistant or the use of the operator's hands,
wherein actuating the switch device causes the automated driver device to move the spring arms or housing to push the spring arms out of the housing and open the the spring arms, and to pull the spring arms into the housing to close the spring arms or by pushing housing over the spring arms, which causes the spring arms to be pushed closed by interior walls of the housing.

4. The operative instrument according to claim 3, wherein the end effector is disposed within a distal end of the endoscope in the instrument channel, and wherein the automated driver device is connected to the end effector through the instrument channel within the endoscope or attached to the outside of the endoscope.

5. The operative instrument according to claim 3, further comprising a removable and replaceable shaft, wherein the automated driver device is connected to said end effector through said removable and replaceable actuator shaft.

6. The operative instrument according to claim 3, wherein the automated driver device is separable from the endoscope and the end effector, and is reusable.

7. The operative instrument according to claim 3, wherein the end effector is separable from the endoscope and is replaceable.

8. The operative instrument according to claim 3, wherein the automated driver device is disposed within the endoscope handle and the end effector is disposed within a distal endoscope section and is removable and replaceable, and wherein the driver device is connected to the end effector via a wire passing down the endoscope shaft to the end effector.

9. The operative instrument according to claim 3, wherein the end effector is attached to a cover of the endoscope and is removable and replaceable, and wherein the automated driver device is connected to the end effector by a wire passing down an exterior of the endoscope shaft to the end effector.

10. The operative instrument according to claim 3, wherein the end effector is disposed outside the endoscope and is connected to the automated driver device via a wire running through the endoscope shaft.

11. The operative instrument according to claim 3, wherein the automated driver device is incorporated into a distal section of the endoscope and the end effector is attached outside of the endoscope and is removable and replaceable.

12. The operative instrument according to claim 3, wherein the automated driver device is attached to an outside of the endoscope.

13. The operative instrument according to claim 3, wherein the switch mechanism is selected from the group consisting of a foot switch, a switch on the shaft or handle, and a voice activated switch.

14. The operative instrument according to claim 3, further comprising a removable and replaceable biopsy cassette mounted within the end effector such that biopsies collected by the end effector are automatically fed into the biopsy cassette in the order of collection.

15. The operative instrument according to claim 3, wherein the automated driver device is selected from the group consisting of a spring(s), electrical solenoid, electric motor, air or hydraulic pressure activated piston, gear(s), and a combination thereof.
